(19) United States
(12) Patent Application Publication
(10) Pub. No.: US 2013/0189781 A1
(43) Pub. Date: Jul. 25, 2013

(54) LOSS OF DE NOVO DNA METHYLTRANSFERASES PROMOTES EXPANSION OF NORMAL HEMATOPOIETIC STEM CELLS

(71) Applicants: Margaret A. Goodell, Houston, TX (US); Grant A. Challen, Houston, TX (US)

(72) Inventors: Margaret A. Goodell, Houston, TX (US); Grant A. Challen, Houston, TX (US)

(21) Appl. No.: 13/577,688

(22) PCT Filed: Feb. 8, 2011
(86) PCT No.: PCT/US2011/024028

§ 371 Date: Jan. 9, 2013

Related U.S. Application Data
(60) Provisional application No. 61/302,398, filed on Feb. 8, 2010.

(51) Int. Cl. C12N 5/0789

(52) U.S. Cl. CPC C12N 5/0647
USPC 435/375

(57) ABSTRACT

Methods and compositions related to increasing expansion of stem cells, such as hematopoietic stem cells (HSCs), via inhibition of at least one of two de novo DNA methyltransferase enzymes, DNMT3a and/or DNMT3b are disclosed.

### CROSS-REFERENCE TO RELATED APPLICATIONS

This application claims the benefit of U.S. Provisional Application 61/302,398 filed on Feb. 8, 2010.

### STATEMENT REGARDING FEDERALLY SPONSORED RESEARCH OR DEVELOPMENT

This invention was made with government support under Grant Numbers K99 DK084259, HL081007, EB005173, and DK58192 awarded by the National Institutes of Health. The government has certain rights in the invention

### TECHNICAL FIELD

The field of the invention includes at least molecular biology, cell biology, hematology, and, in particular aspects, stem cell biology.

### BACKGROUND OF THE INVENTION

Hematopoietic stem cells (HSCs) reside in the bone marrow and cycle through stages of self-renewal, quiescence, proliferation and differentiation to generate all the cell types of the hematopoietic system (Spangrude and Johnson, 1990; Osawa et al., 1996; Venezia et al., 2004; Wilson et al., 2007). It is becoming increasingly apparent that regulation of the stem cell state is modulated by many epigenetic factors and that many important HSC functions are epigenetically pre-programmed. DNA methylation is one of the major epigenetic modifications in the vertebrate genome and has been shown to be a mechanism regulating tissue-specific and/or context-specific gene expression (Attwood et al., 2002). DNA methylation is catalyzed by a family of enzymes called DNA methyltransferases, comprised of the three members—Dnmt1, Dnmt3a and Dnmt3b (Okano et al., 1998). Mouse knock-out mutants have demonstrated that these genes are essential for normal embryonic development with death occurring from the 10-somite stage to 4-weeks postnatally depending on which Dnmt gene is inactivated and how completely DNA (Okana et al., 1999). Dnmt1 preferentially targets hemi-methylated DNA and is principally thought of as a maintenance methyltransferase that re-establishes methylation marks during DNA replication (Lei et al., 1996), although recent reports suggest Dnmt1 has specific functions in HSCs. Dnmt3a and Dnmt3b act as de novo methyltransferases that modify unmethylated DNA (Okana et al., 1999). Embryonic stem (ES) cells that lack Dnmt3a and Dnmt3b are viable and maintain replication potential but progressively lose differentiation potential with repeated passage (Chen et al., 2003). This suggests that these enzymes progressively establish DNA methylation status during differentiation of each lineage to restrict differentiation potential. In HSCs, de novo DNA methylation was implicated to be essential for HSC self-renewal as compound conditional inactivation of both Dnmt3a and Dnmt3b in HSCs lead to inability to sustain long-term hematopoiesis (Tadokoro et al., 2007).

### BRIEF SUMMARY OF THE INVENTION

The present invention is directed to a system and method that regards de novo DNA methylation that balances hematopoietic stem cell (HSC) self-renewal and differentiation. In certain aspects of the invention, methods of the invention encompass loss of de novo DNA methyltransferases that promotes expansion of normal hematopoietic stem cells (expansion encompasses the production of many cells from a single cell).

In some embodiments of the invention, there is a method of augmenting the production of hematopoietic stem cells comprising the down-regulation of de novo methyltransferases.

In some embodiments, there is a method for enhancing the self-renewal capacity of hematopoietic stem cells comprising suppressing the activity of a de novo DNA methyltransferase. In specific embodiments, the de novo DNA methyltransferase is Dmnt3a, Dmnt3b, or both.

In certain aspects of the invention, there is a method for increasing the number of hematopoietic stem cells relative to an unmanipulated control subject comprising suppressing the activity of a de novo DNA methyltransferase, such as Dmnt3a, Dnmt3b, or both, for example.

In embodiments of the invention, there is a method of enhancing proliferation of stem cells, such as hematopoietic stem cells, comprising the step of downregulating DNA methyltransferases (DNMTs) in the cells.

In specific embodiments of the invention, the methods apply to any cell such that inhibiting a DNA methyltransferase results in enhanced proliferation of the cells. In particular aspects, the cells are stem cells, and in certain embodiments the cells have increased expression of the respective DNA methyltransferase(s) compared to another cell. In specific embodiments the stem cells are of any type, including hematopoietic stem cells. In specific embodiments, somatic stem cells are employed in the invention. In particular aspects, the present invention encompasses stem cells of the skin, brain, muscle, liver, gut, brain, heart, mammary tissue, or prostate, for example.

In some embodiments of the invention, there is a method of increasing expansion of stem cells, comprising the step of delivering to the cells an agent that inhibits expression of one or more DNA methyltransferases (DNMT) in the cells. In specific embodiments, the stem cells are hematopoietic stem cells. In some cases, the method is further defined as providing one or more inhibitors of DNMTs to the cells. In specific aspects, the DNMT is DNMT3a, DNMT3b, or both. In particular embodiments, the agent is a nucleic acid, protein (such as an antibody or dominant negative mutant of DNMT3a or DNMT3b), or small molecule. In a specific embodiment, the nucleic acid is siRNA, shRNA, or miRNA. In certain aspects, the agent is selected from the group consisting of RG 108, zebularine, decitabine, 5-azacytidine, BIX01294, 5-azadeoxycytidine, (−)-epigallocatechin-3-gallate (EGCG), 4-aminobenzoic acid derivative, a psammaplin, and an oligonucleotide.

In specific embodiments of the invention, inhibition of DNA methyltransferase(s) in the HSC cells (for example) generates more peripheral blood cells than control HSCs, and in specific embodiments, the increase is at least 2-fold, 3-fold, 4-fold, 5-fold, 6-fold, 7-fold, 8-fold, 9-fold, 10-fold, 15-fold, 20-fold, 25-fold, 30-fold, 50-fold, or greater levels over HSC cells lacking the inhibition. In particular embodiments, the activity of the inhibited HSCs is at least 2-fold, 3-fold, 4-fold, 5-fold, 6-fold, 7-fold, 8-fold, 9-fold, 10-fold, 15-fold, 20-fold, 25-fold, 30-fold, 50-fold, or greater levels compared to controls.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 shows an exemplary expression pattern of de novo DNA methyltransferases Dnmt3a and Dnmt3b in mouse hematopoietic cells. A) Microarray expression profile analysis of myeloid-biased lower-SPKLS HSCs (My-HSCs) and lymphoid-biased upper-SPKLS HSCs (Ly-HSCs) demonstrated that Dnmt3a is more highly expressed in My-HSCs while Dnmt3b is more highly expressed in Ly-HSCs. This was confirmed independently by real-time PCR. (B) Analysis of Dnmt3 expression levels across a spectrum of hematopoietic cell types revealed that both Dnmt3a and Dnmt3b are much more highly expressed in HSCs compared to differentiated blood cells and even closely related hematopoietic progenitors, using real-time PCR. (C) Immunofluorescence co-staining for Dnmt3a and Dnmt3b proteins in purified My-HSCs, Ly-HSCs, short-term HSCs (ST-HSCs) and multipotent progenitors (MPPs) show that protein expression correlates with transcript expression levels. Image analysis determined that the differences in protein expression levels were statistically significant (right panels). (D) High-resolution confocal microscopy of rare HSCs that expressed comparable levels of both DNA methyltransferases revealed that these two proteins rarely co-localize in HSCs, suggesting distinct genomic targets. (E) Sorting of individual HSCs which have retrovirally enforced expression of the Dnmt3s into wells of 96-well plates showed that HSCs transduced with either Dnmt3 had decreased hematopoietic colony forming potential compared to control MSCV-GFP transduced HSCs.

FIG. 2 shows secondary transplantation of Dnmt3 conditional knockout HSCs. (A) Overall contribution to the peripheral blood of KO vs. WT HSCs after secondary transplantation. (B) Analysis of the lineage distribution of Dnmt3 knockout HSCs by analysis of their peripheral blood progeny demonstrated that, compared to their respective control HSCs, Dnmt3a knockout HSCs were slightly more biased towards myeloid cell differentiation while Dnmt3b knockout HSCs show greater propensity for B-cell generation. Both Dnmt3 knockout HSCs showed a mild deficit in T-cell generation compared to control HSCs.

FIG. 3 shows analysis of the HSC compartment of secondary transplant mice. 20-weeks post-secondary transplant, recipient mice were sacrificed and their bone marrow harvested for HSC analysis. Mice transplanted with the knockout HSCs showed a massive increase in the percentage of side population (SP) cells in their bone marrow (top panel). Moreover, virtually all of the SP cells were of donor origin (CD45.2+), demonstrating they were the progeny of transplanted Dnmt3 knockout HSCs (second panel). This is compared to control mice whose SP compartment was generated proportionally by the donor HSCs reflected by their overall level of contribution to the peripheral blood. These expanded HSCs bear all the phenotypic hallmarks of wild-type HSC such as expression of the canonical HSC marker Sca-1 and lack of expression of mature hematopoietic lineage markers, and high expression of the CD150 marker (bottom panel).

FIG. 4 shows that DNA methyltransferases are highly expressed in hematopoietic stem cells and have functional significance. a, Real-time PCR analysis showing Dnmt3a and Dnmt3b mRNA highly and specifically expressed in HSCs. b, Co-immunofluorescent staining of purified cell populations showed Dnmt3a (red) and Dnmt3b (green) proteins were also highly expressed in HSCs compared to ST-HSCs and MPPs. c, Quantification of protein expression levels showed a significant increase in Dnmt3a and Dnmt3b protein in HSCs (n>50 individual cells for each cell type). d, Enforced retroviral over-expression of Dnmt3a and Dnmt3b reduced the colony-forming potential of HSCs in vitro. Data represent two individual experiments each performed with duplicate plates. e, Bone-marrow transplantation of transduced HSCs showed a significant decrease in the peripheral blood progeny of Dnmt3 over-expressing cells (CD45.2⁺GFP⁺), showing alteration of Dnmt3 levels in HSCs has functional consequences in vitro and in vivo. Peripheral blood data are normalized to 4-week engraftment level in each recipient to account for different retroviral transduction levels and data represent two individual experiments with >13 recipient mice in each group.

FIG. 5 shows that conditional deletion of Dnmt3 alleles in HSCs causes a cell-autonomous alteration of HSC activity with serial transplantation. a, Serial competitive transplantation of Dnmt3-KO HSCs showed no differences in engraftment in primary recipients, but enhanced peripheral blood cell generation from Dnmt3a-KO and Dnmt3b-KO HSCs in secondary recipients and beyond. Dnmt3ab-dKO HSCs showed minimal peripheral blood cell generation after the primary transplant. b, Analysis of the proportional hematopoietic lineage differentiation showed that all Dnmt3-KO HSCs became biased towards B-cell production at the expense of T-cell generation with this bias becoming more exacerbated after each round of serial transfer (ND=not determined for Dnmt3ab-dKO HSC-transplanted mice due to lack of peripheral blood cells). c, Analysis of the bone marrow of transplanted mice 18-weeks post-transplant to determine donor-(CD45.2⁺) and competitor-(CD45.1⁺) derived HSCs (SP^{KLS}) showed a significant increase in the total number donor-derived Dnmt3-KO HSCs in serially-transplanted mice. Significantly different values are shown where indicated relative to control mice. Data are representative of at least three individual transplants for each stage of serial transfer with >18, >25, >13 and >11 recipient mice per genotype at primary, secondary, tertiary and quaternary transplant respectively (in most cases many more).

FIG. 6 shows that expansion of Dnmt3-KO HSCs in secondary transplants does not cause increased progenitor cell numbers and is not due to enhanced proliferation or more resistance to apoptosis. a, Hoechst staining and flow cytometric analysis of the bone marrow of secondarily-transplanted mice showed a massive expansion of HSCs in mice re-transplanted with primary Dnmt3a-KO and Dnmt3b-KO HSCs. The SP is indicated by the region in the upper panel. Gating of these HSCs showed virtually all were CD45.2⁺ (lower panel), thus being derived from Dnmt3-KO HSCs. b, Expanded Dnmt3-KO HSC pool in secondary transplants are phenotypically normal sharing HSC markers found on control HSCs (c-Kit⁺ Sca-1⁺ CD150⁺ CD34⁻). c, Dnmt3-KO HSC expansion was consistent between various HSC analysis schemes such as CD150⁺CD48⁻KLS and CD34⁻Flk2⁻KLS. d, Quantification of HSC frequency in bone marrow of secondary-transplanted mice by various HSC phenotypes (shaded area of bar indicates the contribution of donor-derived cells (CD45.2⁺) to the total). e-f, Expanded HSC pools in Dnmt3⁻¹KO transplanted mice did not correlate with an expansion in the different progenitor compartments (shaded area of bar indicates the contribution of donor-derived cells (CD45.2⁺) to the total). g, Transplanted Dnmt3-KO HSCs are less proliferative than transplant-matched control HSCs by quantification of 12-hour BrdU uptake (left), percentage Ki67⁺ HSCs at any given point in time (middle) or propidium iodide staining to reveal the percentage of HSCs not in G₀ (right). h, No difference in apoptotic rate was observed between secondary-transplanted Dnmt3-KO and control HSCs (although all secondary transplanted HSCs were significantly less apoptotic than untransplanted WT HSCs=Un). Data representative of three separate secondary transplant cohorts, n=6-16 mice for each specific phenotypic analysis.

FIG. 7 demonstrates that serial transplantated Dnmt3-KO HSCs are biased towards self-renewal over differentiation in vivo and Dnmt3-KO HSCs have higher expression of “HSC genes”. a, Quantification of self-renewal versus differentiation of serially transplanted control and Dnmt3-KO HSCs. b, Dnmt3-KO HSCs lose differentiation capacity more quickly than control HSCs over serial transplantation while simultaneously displaying much higher self-renewal. c, Microarray global expression profiling of secondarily-transplanted HSCs determined Dnmt3-KO HSCs have higher expression of “HSC fingerprint genes”, that is genes having specific expression in HSCs but not differentiated hematopoietic cell types.

FIG. 8 demonstrates expression patterns of Dnmt3a and Dnmt3b in HSC subtypes. a, Real-time PCR quantification showing Dnmt3a is more highly expressed in myeloid-biased HSCs (My-HSCs; Lower-SP^{KLS}CD150⁺) while Dnmt3b is more highly expressed in lymphoid-biased HSCs (Ly-HSCs; Upper-SP^{KLS}CD150⁺). b, Co-immunofluorescence staining of Dnmt3a (red) and Dnmt3b (green) of purified My-HSCs and Ly-HSCs. Plots under images show quantification of protein expression levels reflecting real-time PCR transcript expression data (n>50 individual cells for each cell type). c, Plot of protein expression of Dnmt3a and Dnmt3b in individual HSCs showing a bimodal distribution of the two HSC subtypes. d, High resolution deconvolution microscopy image showing Dnmt3a and Dnmt3b rarely co-localize in HSCs, indicating each has specific methylation targets.

FIGS. 9A and 9B demonstrate conditional ablation of Dnmt3a and Dnmt3b in the hematopoietic system does not affect steady-state adult hematopoiesis. Peripheral blood counts of adult Mx1-cre; Dnmt3a^{fl/fl} and Mx1-cre; Dnmt3b^{fl/fl} mice (cre−=control; cre+=experimental) following sequential pIpC injections (shaded red areas) show that loss of these enzymes in the adult does not affect steady-state hematopoiesis in terms of white blood cell (WBC), platelet or red blood cell (RBC) counts or hemoglobin (Hb). When the mice were challenged with 5-fluorouracil (5-FU; blue arrows), there was a measureable response on peripheral blood kinetics but it was the same for control and experimental animals.

FIG. 10 shows loss of Dnmt3 causes a cell-autonomous change in HSC functional output. a, Counting the average number of hematopoietic colonies formed per 96-well Methocult plate from donor-derived HSCs (CD45.2⁺SP^{KLS}CD150⁺) after each stage of serial transplantation showed a significant increase in activity on a per cell basis from Dnmt3-KO HSCs. b, PCR screening for floxed allele deletion in individual Methocult colonies (each arising from a single HSC) revealed a high efficiency of target sequence deletion for Dnmt3a (upper panel) and Dnmt3b (lower panel). In representative Dnmt3b PCR screen, red star indicates a HSC that completely escaped deletion and maintained two wild-type alleles while yellow stars indicate HSCs in which only one allele was deleted (haploinsufficient). Efficiency of floxed allele deletion was >95% in almost all experiments.

### DETAILED DESCRIPTION OF THE INVENTION

As used herein, the use of the word “a” or “an” when used in conjunction with the term “comprising” in the claims and/or the specification may mean “one,” but it is also consistent with the meaning of “one or more,” “at least one,” and “one or more than one.” Some embodiments of the invention may consist of or consist essentially of one or more elements, method steps, and/or methods of the invention. It is contemplated that any method or composition described herein can be implemented with respect to any other method or composition described herein.

As used herein, the term “DNA methyltransferase” family of enzymes catalyze the transfer of a methyl group to DNA. Three active DNA methyltransferases have been identified in mammals, including DNMT1, DNMT3A and DNMT3B. In alternative embodiments, the present invention encompasses DNMT1 and/or DNMT3L.

As used herein, the term “hematopoietic stem cell” refers to a cell that can generate most types of differentiated peripheral blood cells, and can generate more stem cells. These cells are usually defined by their activity in a bone marrow transplantation assay, or their phenotype according to cell surface markers. In exemplary but merely specific embodiments, mouse HSCs are defined as cells displaying the side population (SP) phenotype, c-Kit⁺Lin⁻Sca-1⁺ (SP^{KLS}), although in certain aspects they could also be c-kit⁺, Sca-1⁺, and CD150⁺. In human HSCs, the HSCs cells would be CD34+CD38−.

### I. General Embodiments of the Invention

In certain embodiments of the invention, there are methods and compositions that regard increased expansion of mammalian cells, including stem cells, for example hematopoietic stem cells; in alternative embodiment, other types of stem cells are subjected to methods of the invention, including stem cells of the skin, neuron, muscle, liver, gut, brain, heart or mammary tissue, for example. The cells are modified to exhibit increased expansion compared to other cells of the same type that have not been subjected to the same modification. In particular aspects, the cells are subjected to inhibition of a DNA methyltransferase, although in alternative embodiments there is inhibition of more than one DNA methyltransferase. In certain embodiments, however, the cells are subjected to inhibition of one DNA methyltransferase but not subjected to inhibition of one or more other particular DNA methyltransferase(s). In some cases, the methods of the present invention encompass inhibition of DNMT3A but not DNMT3B, or vice versa.

In specific embodiments of the invention, the DNA methyltransferase that is inhibited is DNMT3A. DNMT3A may also be referred to as DNA (cytosine-5-)-methyltransferase 3 alpha; DNA cytosine methyltransferase 3A2; DNA methyltransferase HsaIIIA; OTTHUMP000002011492; M.HsaIIIA; DNA (cytosine-5)-methyltransferase 3A; DNA MTase HsaIIIA; Dnmt3a; DNMT3A2; EC 2.1.1.37; or OTTHUMP000002011502, for example

In some embodiments, the DNA methyltransferaseDNMT3B may also be referred to as DNA (cytosine-5-)-methyltransferase 3 beta; DNA (cytosine-5-)-methyltransferase 3 beta; ICF; M.HsaIIIB; DNA (cytosine-5)-methyltransferase 3B; DNA MTase HsaIIIB; EC 2.1.1.37; DNA methyltransferase HsaIIIB; Dnmt3b, for example.

Embodiments of the present invention also include cells generated by methods of the present invention, including generated by the inhibition of DNA methyltransferase. Certain embodiments of the present invention include methods of making the DNA methyltransferase-inhibited cells. Kits of the invention for making or using the DNA methyltransferase-inhibited cells are also envisioned. Inhibitors of DNA methyltransferase are included in embodiments of the invention.

### II. Inhibitors of DNA Methyltransferases

In embodiments of the invention, DNA methyltransferase inhibitors are employed to facilitate expansion of stem cells.

Inhibitors may be of any kind, but in specific embodiments they are nucleic acid, protein, or small molecule, for example. Exemplary nucleic acids include those for RNAi, including siRNA or shRNA. Exemplary proteins include antibodies (Sigma-Aldrich®, for example) or dominant-negative versions of the DNMT3A or DNMT3B proteins.

Exemplary DNA methyltransferase inhibitors that may be used in the invention include the following: RG 108, Zebularine, Decitabine, 5-Azacytidine, BIX01294, 5-azadeoxycytidine, (−)-epigallocatechin-3-gallate (EGCG), 4-Aminobenzoic acid derivatives (such as the antiarrhythmic drug procainamide and the local anesthetic procaine), psammaplins (for example, Psammaplin A, D, E, F, G, H, and/or I), oligonucleotides (including hairpin loops and specific antisense oligonucleotides, such as MG98), or a combination thereof.

Exemplary DNMT3A GenBank® sequences, incorporated by reference herein, are as follows: NM_{—}022552.3 NM_{—}153759.2 NM_{—}175629.1 NM_{—}175630.1. The skilled artisan recognizes that inhibition of these or similar sequences, for example, may be employed in methods of generating cells having increased expansion.

Exemplary DNMT3B GenBank® sequences, incorporated by reference herein, are as follows: NM_{—}006892.3 NM_{—}175848.1 NM_{—}175849.1 NM_{—}175850.1. The skilled artisan recognizes that inhibition of these or similar sequences, for example, may be employed in methods of generating cells having increased expansion.

In some embodiments of the invention, the respective DNA methyltransferase is inhibited with the employment of microRNAs. Exemplary microRNAs that regulate DNMT3A are as follows: hsa-miR-30c, hsa-miR-429, hsa-miR-29c, hsa-miR-29a, hsa-miR-30d, hsa-miR-218, hsa-miR-410, hsa-miR-132, hsa-miR-30a, hsa-miR-144, hsa-miR-383, hsa-miR-212, hsa-miR-96, hsa-miR-370, hsa-miR-200c, hsa-miR-182, hsa-miR-143, hsa-miR-101, hsa-miR-30b, hsa-miR-194, hsa-miR-29b, and hsa-miR-30e (SABiosciences).

Exemplary microRNAs that regulate DNMT3B are as follows: hsa-miR-203, hsa-miR-370, hsa-miR-29c, hsa-miR-29a, and hsa-miR-29b (SABiosciences).

### III. Nucleic Acid-Based Expression Systems

In embodiments of the invention, aspects include use of expression vectors to generate inhibition of DNA methyltransferase in stem cells, including HSCs. Aspects of this embodiment includes vectors, particularly for mammalian cell transformation. The promoter in particular embodiments is preferably operative in a eukaryotic cell.

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

1. Promoters and Enhancers

In certain aspects, a vector employed in generation of mammalian cells having inhibited DNA methyltransferase is further defined as a vector having regulatory elements, such as promoters and, optionally, enhancer. A skilled artisan recognizes that a variety of known mammalian promoters in the art are known (see, for example, MPromDb (Mammalian Promoter Database) on the world wide web).

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30 110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the □ lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base, EPDB) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference.)

5. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

7. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

8. Selectable and Screenable Markers

In certain embodiments of the invention, cells containing a nucleic acid construct of the present invention may be identified in vitro or in vivo by including a marker in the expression vector, for example to identify cells wherein they have been transformed to encompass inhibition of DNA methyltransferase. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEMTM 11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S transferase (GST) soluble fusion proteins for later purification and separation or cleavage. Other suitable fusion proteins are those with beta galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

C. Viral Vectors

The ability of certain viruses to infect cells or enter cells via receptor mediated endocytosis, and to integrate into host cell genome and express viral genes stably and efficiently have made them attractive candidates for the transfer of foreign nucleic acids into cells (e.g., mammalian cells). Embodiments of the present invention may include a viral vector that encodes an inhibitor of DNA methyltransferase. Non-limiting examples of virus vectors that may be used to deliver a nucleic acid of the present invention are described below.

1. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell specific construct that has been cloned therein. Knowledge of the genetic organization or adenovirus, a 36 kb, linear, double stranded DNA virus, allows substitution of large pieces of adenoviral DNA with foreign sequences up to 7 kb (Grunhaus and Horwitz, 1992).

2. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno associated virus (AAV) is an attractive vector system for use in the present invention as it has a high frequency of integration and it can infect nondividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

3. Retroviral Vectors

Retroviruses are useful as delivery vectors because of their ability to integrate their genes into the host genome, transferring a large amount of foreign genetic material, infecting a broad spectrum of species and cell types and of being packaged in special cell lines (Miller, 1992).

In order to construct a retroviral vector housing an inhibitor of DNA methyltransferase, a nucleic acid (e.g., one encoding an RNAi of DNMT3a or DNMT3b) is inserted into the viral genome in the place of certain viral sequences to produce a virus that is replication defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

D. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

E. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

F. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations, so long as the progeny cells still encompass inhibition of DNA methyltransferase. In the context of expressing a heterologous nucleic acid sequence, “host cell” refers to a eukaryotic cell, and it includes any transformable organism that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny. As used herein, the terms “engineered” and “recombinant” cells or host cells are intended to refer to a cell into which an exogenous nucleic acid sequence, such as, for example, a vector, has been introduced. Therefore, recombinant cells are distinguishable from naturally occurring cells that do not contain a recombinantly introduced nucleic acid.

In certain embodiments, it is contemplated that RNAs or proteinaceous sequences may be co expressed with other selected RNAs or proteinaceous sequences in the same host cell. Co expression may be achieved by co transfecting the host cell with two or more distinct recombinant vectors. Alternatively, a single recombinant vector may be constructed to include multiple distinct coding regions for RNAs, which could then be expressed in host cells transfected with the single vector.

In certain embodiments, the host cell or tissue may be comprised in at least one organism. In certain embodiments, the organism may be, but is not limited to, a prokayote (e.g., a eubacteria, an archaea) or an eukaryote, as would be understood by one of ordinary skill in the art.

Numerous cell lines and cultures are available for use as a host cell, and they can be obtained through the American Type Culture Collection (ATCC), which is an organization that serves as an archive for living cultures and genetic materials. An appropriate host can be determined by one of skill in the art based on the vector backbone and the desired result. A plasmid or cosmid, for example, can be introduced into a prokaryote host cell for replication of many vectors.

Examples of eukaryotic host cells for replication and/or expression of a vector include, but are not limited to, stem cells. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

### IV. Transformation of Mammalian Cells

In some embodiments of the invention, cells having a state of inhibition of DNA methyltransferase are employed that have increased proliferation compared to the same stem cells lacking inhibition of the DNA methyltransferase.

Suitable methods for nucleic acid delivery for transformation of a mammalian cell are well known in the art and include virtually any method by which a nucleic acid (e.g., DNA or nucleic acid for RNAi) can be introduced into the cell. Such methods include, but are not limited to, direct delivery of DNA such as by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985), and any combination of such methods. Through the application of techniques such as these, cell(s) may be stably or transiently transformed.

A. Injection

Certain embodiments of the present invention include the introduction of a nucleic acid by direct microinjection. Direct microinjection has been used to introduce nucleic acid constructs into Xenopus oocytes (Harland and Weintraub, 1985). The amount of DNA methyltransferase inhibitor (for nucleic acid aspects, for example) used may vary upon the nature of the antigen as well as the organelle, cell, tissue or organism used

B. Electroporation

In certain embodiments of the present invention, a nucleic acid is introduced into an a cell via electroporation. Electroporation involves the exposure of a suspension of cells and DNA to a high voltage electric discharge. In some variants of this method, certain cell wall degrading enzymes, such as pectin degrading enzymes, are employed to render the target recipient cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre B lymphocytes have been transfected with human kappa immunoglobulin genes (Potter et al., 1984), and rat hepatocytes have been transfected with the chloramphenicol acetyltransferase gene (Tur Kaspa et al., 1986) in this manner.

C. Calcium Phosphate

In other embodiments of the present invention, a nucleic acid is introduced to the cells using calcium phosphate precipitation. Human KB cells have been transfected with adenovirus 5 DNA (Graham and Van Der Eb, 1973) using this technique. Also in this manner, mouse L(A9), mouse C127, CHO, CV 1, BHK, NIH3T3 and HeLa cells were transfected with a neomycin marker gene (Chen and Okayama, 1987), and rat hepatocytes were transfected with a variety of marker genes (Rippe et al., 1990).

D. DEAE Dextran

In another embodiment, a nucleic acid is delivered into a cell using DEAE dextran followed by polyethylene glycol. In this manner, reporter plasmids were introduced into mouse myeloma and erythroleukemia cells (Gopal, 1985).

E. Sonication Loading

Additional embodiments of the present invention include the introduction of a nucleic acid by direct sonic loading. LTK fibroblasts have been transfected with the thymidine kinase gene by sonication loading (Fechheimer et al., 1987).

F. Liposome Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome mediated nucleic acid delivery and expression of foreign DNA in vitro has been very successful (Nicolau and Sene, 1982; Fraley et al., 1979; Nicolau et al., 1987). The feasibility of liposome mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome encapsulated DNA (Kaneda et al., 1989). In other embodiments, a liposome may be complexed or employed in conjunction with nuclear non histone chromosomal proteins (HMG 1) (Kato et al., 1991). In yet further embodiments, a liposome may be complexed or employed in conjunction with both HVJ and HMG 1. In other embodiments, a delivery vehicle may comprise a ligand and a liposome.

G. Receptor Mediated Transfection

Still further, a nucleic acid may be delivered to a target cell via receptor mediated delivery vehicles. These take advantage of the selective uptake of macromolecules by receptor mediated endocytosis that will be occurring in a target cell. In view of the cell type specific distribution of various receptors, this delivery method adds another degree of specificity to the present invention.

Certain receptor mediated gene targeting vehicles comprise a cell receptor specific ligand and a nucleic acid binding agent. Others comprise a cell receptor specific ligand to which the nucleic acid to be delivered has been operatively attached. Several ligands have been used for receptor mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell specific binding. For example, lactosyl ceramide, a galactose terminal asialganglioside, have been incorporated into liposomes and observed an increase in the uptake of the insulin gene by hepatocytes (Nicolau et al., 1987). It is contemplated that the tissue specific transforming constructs of the present invention can be specifically delivered into a target cell in a similar manner.

H. Microprojectile Bombardment

Microprojectile bombardment techniques can be used to introduce a nucleic acid into at least one, organelle, cell, tissue or organism (U.S. Pat. No. 5,550,318; U.S. Pat. No. 5,538,880; U.S. Pat. No. 5,610,042; and PCT Application WO 94/09699; each of which is incorporated herein by reference). This method depends on the ability to accelerate DNA coated microprojectiles to a high velocity allowing them to pierce cell membranes and enter cells without killing them (Klein et al., 1987). There are a wide variety of microprojectile bombardment techniques known in the art, many of which are applicable to the invention.

Microprojectile bombardment may be used to transform various cell(s), tissue(s) or organism(s), such as for example any plant species. Examples of species which have been transformed by microprojectile bombardment include monocot species such as maize (PCT Application WO 95/06128), barley (Ritala et al., 1994; Hensgens et al., 1993), wheat (U.S. Pat. No. 5,563,055, incorporated herein by reference), rice (Hensgens et al., 1993), oat (Torbet et al., 1995; Torbet et al., 1998), rye (Hensgens et al., 1993), sugarcane (Bower et al., 1992), and sorghum (Casas et al., 1993; Hagio et al., 1991); as well as a number of dicots including tobacco (Tomes et al., 1990; Buising and Benbow, 1994), soybean (U.S. Pat. No. 5,322,783, incorporated herein by reference), sunflower (Knittel et al. 1994), peanut (Singsit et al., 1997), cotton (McCabe and Martinell, 1993), tomato (VanEck et al. 1995), and legumes in general (U.S. Pat. No. 5,563,055, incorporated herein by reference).

In this microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

### V. Generation and Use of Transgenic Mice

In embodiments of the invention, transgenic mice are utilized to study the effects of mammals lacking a DNA methyltransferase. The skilled artisan recognizes that it is routine to generate transgenic mice in the art. For example, there are two major methods to generate transgenic mice, including by pronuclear microinjection and introduction of DNA into embryonic stem cells (ES cells).

In pronuclear microinjection, foreign DNA, for example RNAi to inhibit DNMT3a or DNMT3b, is introduced directly into the mouse egg just after fertilization. The DNA is injected into the large male pronucleus (from the sperm) and integrates as many tandemly arranged copies at a random position in the genome, often after one or two cell divisions have occurred. In cases wherein the transgenic cells contribute to the germ line, then some transgenic eggs or sperm are produced and the subsequent generation of mice will be fully transgenic.

Another method includes the introduction of DNA into embryonic stem cells (ES cells) that are derived from the very early mouse embryo that can differentiate into all types of cells when introduced into another embryo. Although some cases allow random integration, other cases include homologous recombination that concerns integration of a single copy at a specific site. The ES cells will colonize a host embryo and in some cases contribute to the germ line, resulting in the production of some sperm carrying the foreign DNA. When these transgenic sperm fertilize a normal egg, a transgenic mouse is produced with the same foreign DNA in every cell.

### VI. Kits of the Invention

Any of the compositions described herein may be comprised in a kit. In a non-limiting example, reagents appropriate for generating and/or culturing cells of the invention may be comprised in a kit. In some embodiments, the reagents useful to prepare compositions for generating the cells disclosed in the invention are included in the kit.

The kits may thus comprise, in suitable container means, one or more of cells, including stem cells, for example HSCs; inhibitors, including DNMT3B or DNMT3A inhibitors; vectors; oligonucleotides; and/or buffer reagents to produce cells in which DNMT3A or DNMT3B was inhibited. In particular embodiments, the kit may include one or more of the following: RG 108, Zebularine, Decitabine, 5-Azacytidine, BIX01294, 5-azadeoxycytidine, (−)-epigallocatechin-3-gallate (EGCG), 4-Aminobenzoic acid derivatives (such as the antiarrhythmic drug procainamide and the local anesthetic procaine), psammaplins (for example, Psammaplin A, D, E, F, G, H, and/or I), oligonucleotides (including hairpin loops and specific antisense oligonucleotides, such as MG98), or a combination thereof

Where appropriate, the kit may comprise suitably aliquoted components. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional component(s) may be separately placed. However, various combinations of components may be comprised in a vial. The kits of the present invention also will typically include a means for containing the components and any other reagent containers in close confinement for commercial sale. Such containers may include injection or blow molded plastic containers into which the desired vials are retained, for example.

When the components of the kit are provided in one and/or more liquid solutions, the liquid solution is an aqueous solution, with a sterile aqueous solution being particularly preferred. However, in some embodiments the components of the kit are provided as dried powder(s). When reagents and/or components are provided as a dry powder, the powder can be reconstituted by the addition of a suitable solvent. It is envisioned that the solvent may also be provided in another container means, in some cases.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Loss of De Novo DNA Methyltransferases Promotes Expansion of Normal Hematopoietic Stem Cells

Hematopoietic stem cells (HSC) have been viewed as a uniform population of cells that can, with equal potency, generate all of the cells of the peripheral blood. Close examination of this tenet by several labs has recently led to the realization that HSC are better viewed as a consortium of stem cells with slightly varied behaviors. All of these HSC can generate all of the blood cells over a period of time, but some do this with greater potency and with somewhat different differentiation proclivities.

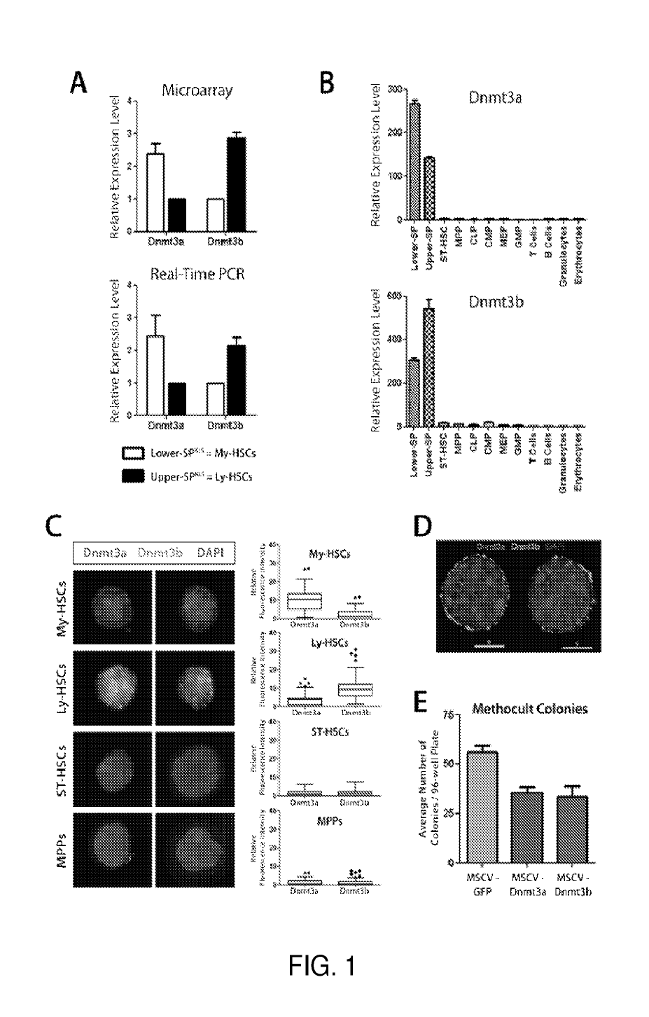

To characterize the mechanisms that control the behavior of these stem cells, the inventors performed gene expression profiling on the two major sub-types of purified HSC. Among other differences, there was a striking distinction in their expression of the de novo DNA methyltransferases. As shown in FIG. 1A, Dnmt3a is specifically expressed in myeloid-biased HSCs (My-HSCs), while Dnmt3b is specific to lymphoid-biased HSCs (Ly-HSCs). The de novo DNA methyltransferases are also highly enriched in the stem cell population relative to progenitors and differentiated cells types (FIG. 1B). Immunohistochemistry verifies this (FIG. 1C). The few stem cells that express both Dnmt3s (the balanced HSCs) show distinct non-overlapping patterns of protein localization (FIG. 1D). Overexpression of the Dnmt3s appears to inhibit colony formation (FIG. 1E).

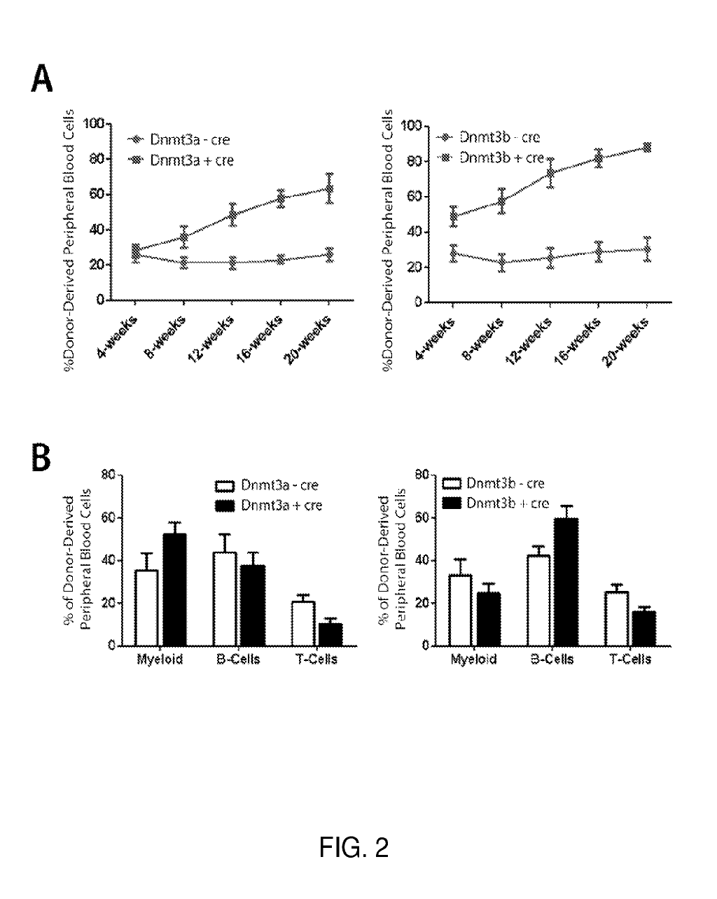

To investigate the function of Dnmt3s in the mouse, the inventors obtained conditional KO mice and used them to study the effect of loss of Dnmt3a and Dnmt3b in mouse HSCs. The inventors crossed mice carrying floxed alleles of Dnmt3a or Dnmt3b to Mx1-cre mice which generate hematopoietic stem cell knockouts after induction of cre expression by pIpC injection. There was no observable defect in the function of either Dnmt3a or Dnmt3b knockout HSCs in primary bone marrow transplantation, the gold standard for in vivo HSC functional activity. However, when the knockout HSCs from the primary recipients were re-purified and transplanted into secondary recipients, a striking trend was noted. The overall engraftment levels, or contribution to peripheral blood generation in the lethally irradiated recipients, were significantly higher from Dnmt3a and Dnmt3b knockout HSCs compared to cre-control HSCs. Interestingly, while both knockout HSC types generated more peripheral blood cells than control HSCs, the Dnmt3b knockout HSCs produced this effect faster, and to much higher overall levels than Dnmt3a. 20-weeks post-transplant, the activity of Dnmt3a- and Dnmt3b-knockout HSCs was approximately 3-fold and 4-fold higher compared to their respective controls (FIG. 2). This is extremely striking. The mice do not appear to have any type of malignancy—the knock-out HSCs are simply outcompeting their normal WT counterparts, by this measure of differentiation. The knock out cells appear to contribute to differentiation of all cell types to a normal level (only mild differentiation biases).

Given their striking expression pattern in HSC subtypes, the inventors characterized whether HSC lineage fate determination is at least partly mediated by the actions of DNA methyltransferase. While the inventors see some lineage skewing in long-term transplanted knockout HSCs, this phenotype is secondary to the observed enhancement in overall peripheral blood cell production.

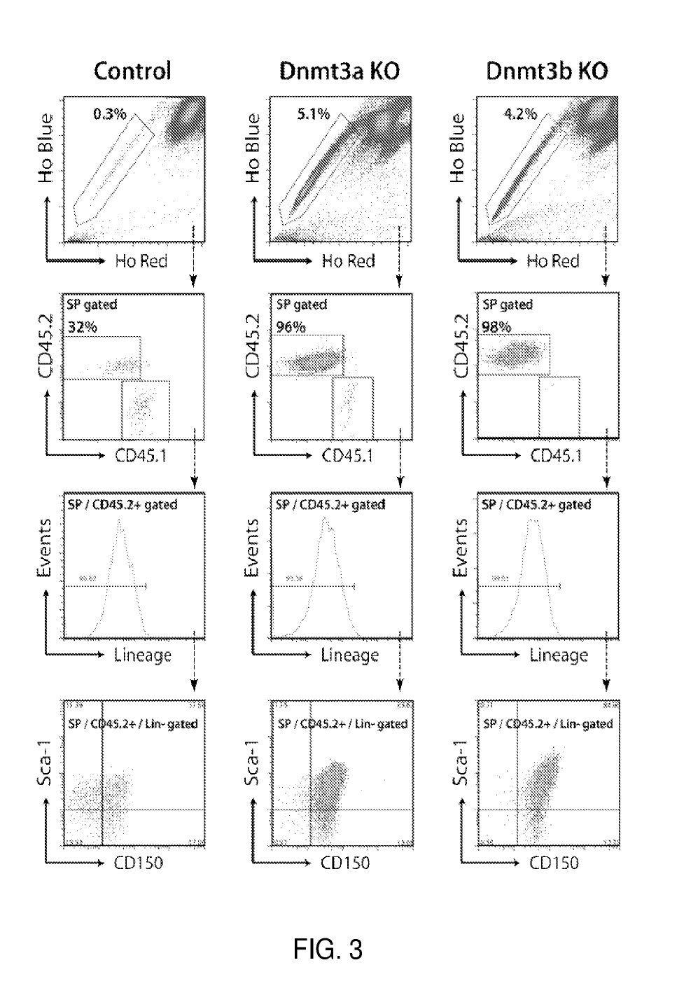

In some embodiments of the invention, the increase in Dnmt3-KO-derived cells in the peripheral blood is a result of increased differentiation capacity, or a direct effect on the HSC in the bone marrow. To examine this, the inventors sacrificed the mice from the studies shown in FIG. 2 and analyzed their bone marrow stem cell compartment. The mice transplanted with Dnmt3 KO cells had an enormous expansion of their HSCs, as defined by multiple markers (FIG. 3). Combined with the observation that the transplanted mice exhibited higher levels of differentiated cells in the blood, this strongly indicates that deletion of Dnmt3a or Dnmt3b increases self-renewal or expansion of normal HSC.

While other attempts at expanding HSC have resulted in apparent expansion of HSC defined solely by phenotype, the inventors are unaware of any protocol that has yielded a similar functional increase in HSC.

In embodiments of the invention wherein HSC expansion may be reversible, one can characterize this by transducing the expanded HSC with Dntm3-expressing retroviruses to see if they return to “normal”. In embodiments of the invention wherein it is determined how much expansion the cells can undergo, one can perform serial transplantation of the HSC to determine whether the cells lose their ability to expand, or to differentiate. In embodiments where the specific mechanisms are determined through which loss of Dnmt3s induces this effect, one can perform molecular analyses of Dnmt3 targets.

In particular embodiments of the invention, there is expansion of “normal” HSC through DNMT3 inhibition, including through transient DNMT3 inhibition. In specific embodiments of the invention, DNMT3 inhibitors have different effects on normal HSC than malignant “stem cells”, which are a great clinical benefit.

### Example 2

### De Novo DNA Methylation Balances Hematopoietic Stem Cell Self-Renewal and Differentiation

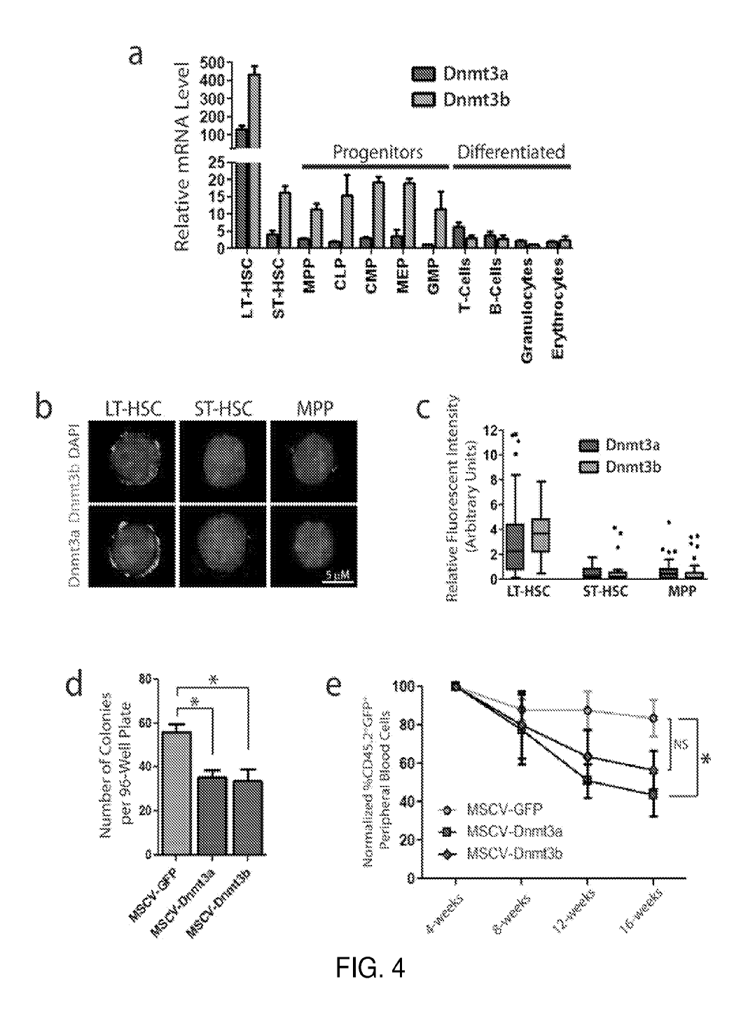

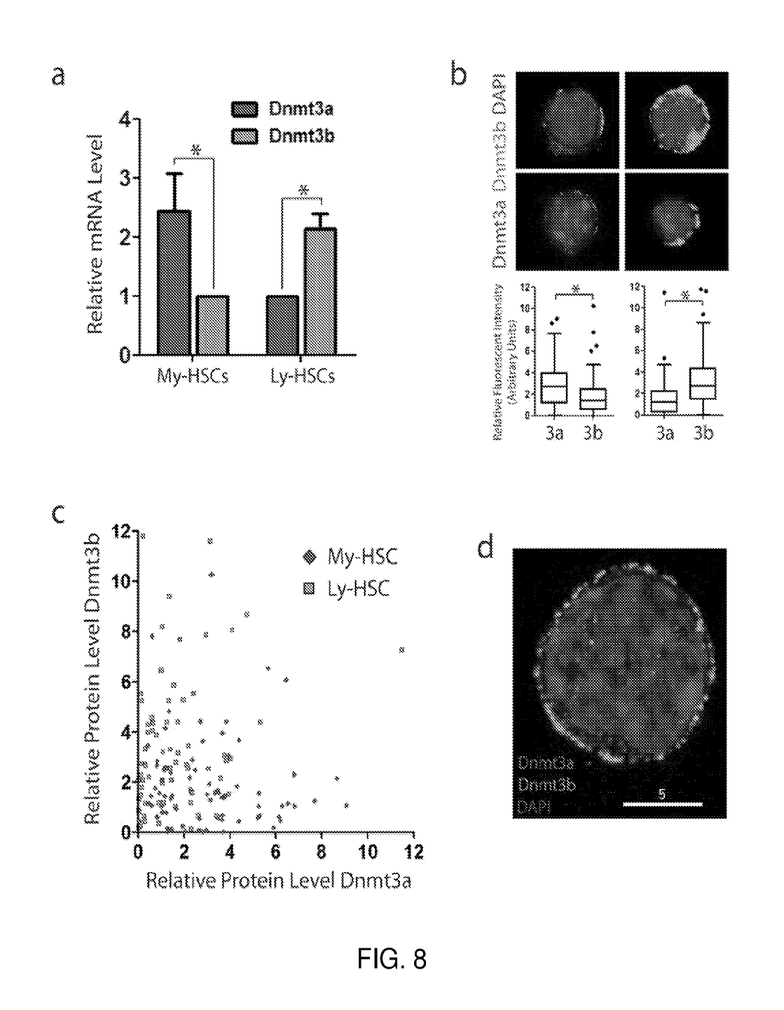

From a previous molecular analysis (Challen et al., 2010), the inventors noted very high transcript expression of Dnmt3a and Dnmt3b in HSCs. Interestingly, they observed some clonal variation in the expression patterns within the HSC pool with myeloid-biased HSCs having higher expression of Dnmt3a and lymphoid-biased HSCs having higher expression of Dnmt3b (FIG. 8a-c). A more comprehensive survey of the hematopoietic system showed that both Dnmt3a and Dnmt3b are highly and specifically expressed in long-term HSCs at both the mRNA and protein levels (FIG. 4a-c), even compared with closely related short-term HSCs (ST-HSCs) and multipotent progenitors (MPPs). The proteins rarely co-localized in HSCs, suggesting that each has specific methylation targets in the HSC genome (FIG. 8d). While a previous study reported that conditional ablation of both Dnmt3a and Dnmt3b resulted in loss of long-term HSC self-renewal capacity (Tadokoro et al., 2007), HSCs lacking only one of these proteins were still capable of long-term hematopoiesis. In certain embodiments of the invention, the inventors considered that Dnmt3a and Dnmt3b empowers different HSCs clones with distinct functional characteristics and that DNA methylation are involved in acute cell fate decisions in HSCs as opposed to the more traditional view of its role in long-term epigenetic gene silencing.

The inventors first assessed the roles of Dnmt3a and Dnmt3b in HSCs in a gain-of-function assay using enforced retroviral over-expression. Cycling HSCs were transduced with the murine stem cell virus (MSCV) containing the full-length open reading frame for either Dnmt3a (MSCV-Dnmt3a) or Dnmt3b (MSCV-Dnmt3b) with tracking of transduced cells by bicistronic expression of GFP. Empty vector (MSCV-GFP) was used in parallel as a control. Over-expression of Dnmt3a and Dnmt3b caused a significant reduction in colony-forming capacity of HSCs in vitro (FIG. 4d), and resulted in reduced GFP+ cells (the progeny of transduced HSCs) in the peripheral blood of recipient mice in transplantation experiments (FIG. 4e). Over-expression of Dnmt3a and Dnmt3b also skewed the lineage differentiation of HSCs towards slightly higher T-cell generation. These data indicated alteration of Dnmt3 levels in HSCs can have functional consequences.

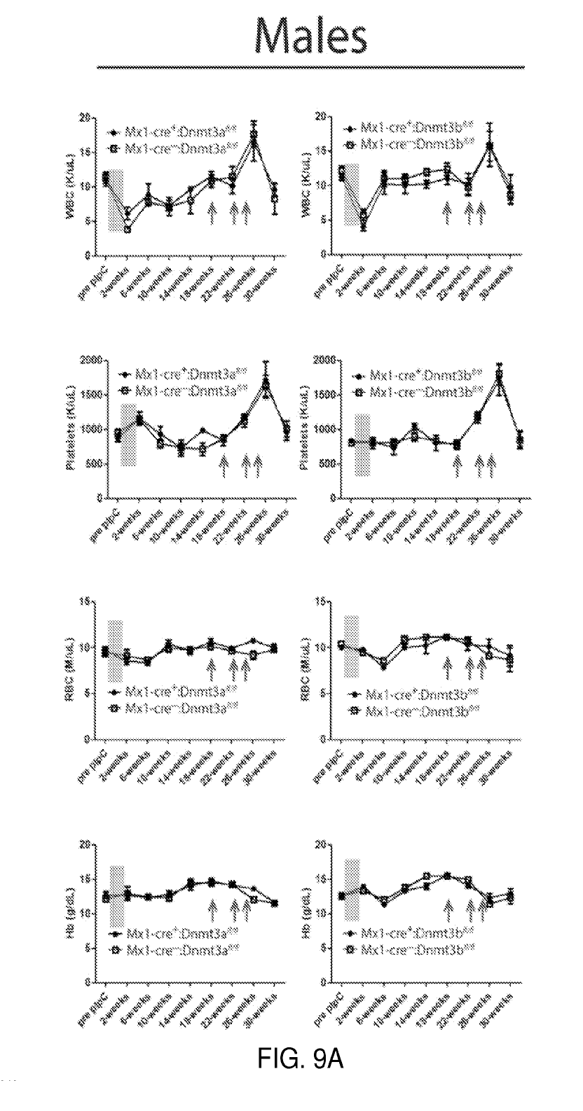

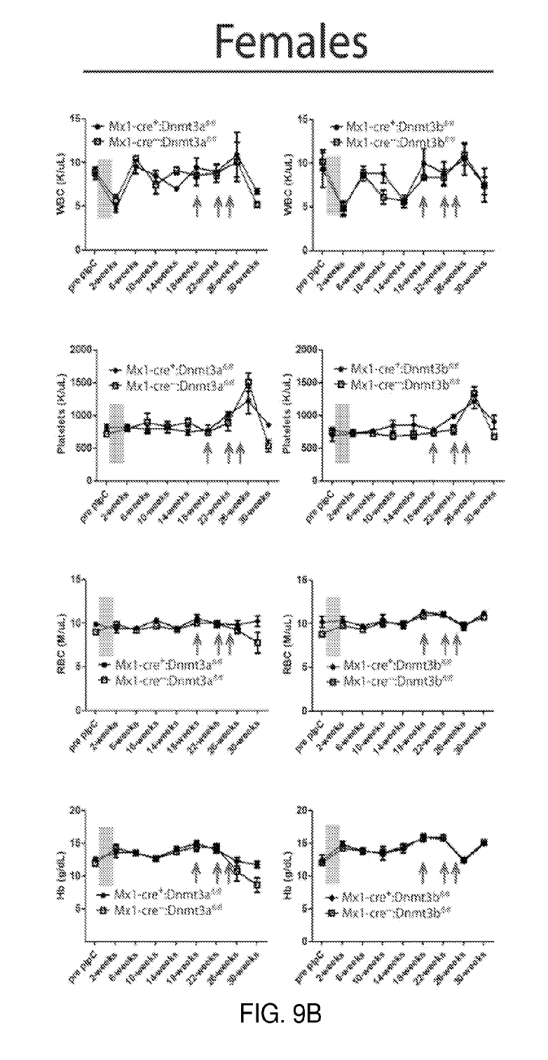

To further investigate the functions of Dnmt3a and Dnmt3b in HSCs, the inventors generated inducible conditional knockout mice by crossing Dnmt3^{afl/fl}, Dnmt3^{bfl/fl} and Dnmt3^{afl/fl}Dnmt3b^{fl/fl} mice (Dodge et al., 2005) with mice carrying the Mx1-cre driver, allowing conditional ablation specifically in the hematopoietic system. Deletion of floxed alleles was mediated by six sequential injections of polyinosinic-polycytidylic acid (pIpC) every other day to induce an interferon response and expression of Mx1. There was no difference in adult hematopoiesis in Mx1-cre⁺; Dnmt3a^{Δ/Δ} (henceforth referred to as Dnmt3a-KO), Mx1-cre⁺; Dnmt3b^{Δ/Δ} (henceforth referred to as Dnmt3b-KO) and Mx1-cre⁺; Dnmt3a^{Δ/Δ}Dnmt3b^{Δ/Δ} (henceforth referred to as Dnmt3ab-dKO) mice compared to gender-matched Mx1-cre-; Dnmt3a^{fl/fl}, Mx1-cre-; Dnmt3b^{fl/fl} and Mx1-cre⁻; Dnmt3a^{fl/fl}Dnmt3b^{fl/fl} littermates (henceforth referred to as control) respectively, even when mice were challenged by the myeloablative agent 5-fluorouracil (FIG. 9).

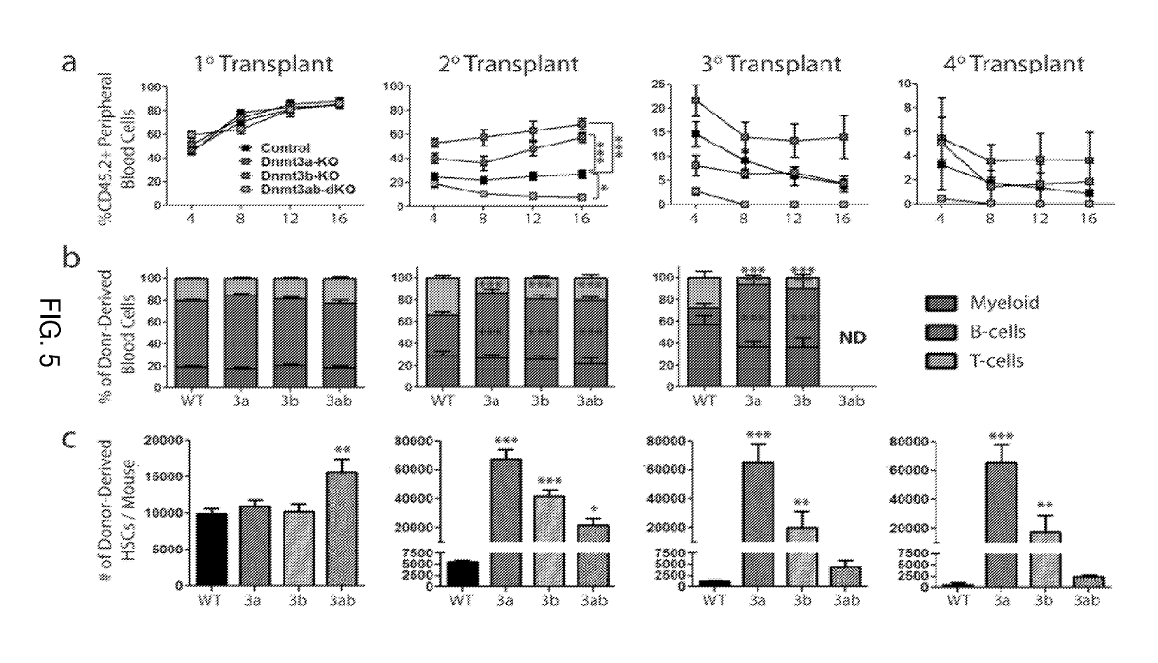

To more specifically interrogate the effect of conditional ablation of Dnmt3s in stem cells, 250 Dnmt3a-KO, Dnmt3b-KO or Dnmt3ab-dKO HSCs (Side Population⁺ c-Kit⁺ Lineage-Sca-1⁺=SP^{KLS}) were transplanted into wild-type lethally irradiated mice in parallel with corresponding Mx1-cre-control HSCs for the respective genotypes (results for control HSCs are compiled in final figures). Conditional deletion of floxed alleles was induced by pIpC injections in the recipient mice 4-weeks post-transplant, thereby specifically ablating floxed alleles in the test donor HSCs, and not affecting wild-type whole bone marrow competitor or recipient cells. There was no difference in peripheral blood chimerism or HSC lineage differentiation between control and Dnmt3-KO HSCs in the absence of other hematopoietic perturbation (FIG. 5). However, when Dnmt3a- and Dnmt3b-KO HSCs (CD45.2+SP^{KLS}) were re-purified from the bone marrow of primary recipients 18-weeks post-transplant and transferred to secondary recipients, they exhibited dramatically higher peripheral blood reconstitution compared to control HSCs (FIG. 5a). Secondarily transplanted Dnmt3ab-dKO HSCs showed a significant decline in peripheral blood contribution, consistent with a previous study reporting that loss of both Dnmt3a and Dnmt3b in HSCs resulted in defective self-renewal (Challen et al., 2010). All secondarily-transplanted Dnmt3-KO HSC genotypes showed skewing of their lineage-differentiation output with a bias towards B-cell generation at the expense of T-cell differentiation (FIG. 5b).

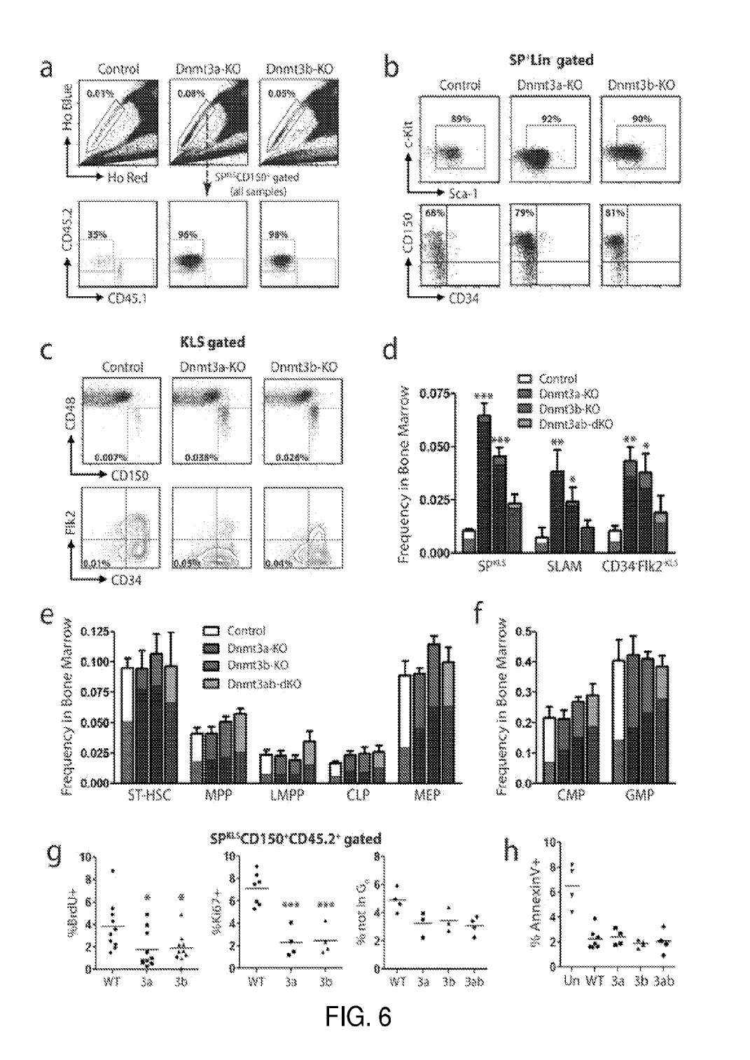

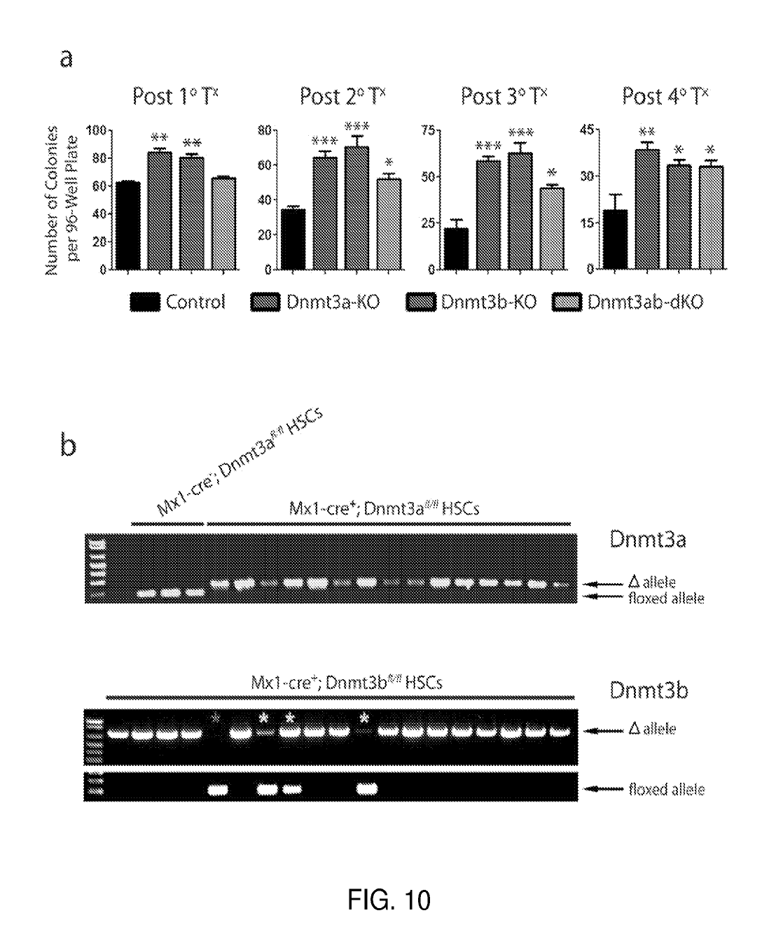

Analysis of the bone marrow of secondary-transplanted mice showed that the increase in peripheral blood cell generation from Dnmt3a- and Dnmt3b-KO HSCs corresponded with a >4-fold expansion of the phenotypically-defined HSC pool, with virtually all of these being derived from the Dnmt3a- and Dnmt3b-KO donor HSCs (CD45.2⁺; FIG. 5c). This increase in HSC abundance was consistent regardless of the phenotypic criteria used, including SP^{KLS}, SLAM (CD48-CD150⁺KLS) or CD34-Flk2-KLS (FIG. 6a-d). Despite significantly lower contribution to peripheral blood cell differentiation, the bone marrow of mice secondarily transplanted with Dnmt3ab-dKO HSCs also showed a modest expansion of the HSC pool (˜2-fold) compared to control, again with the vast majority of these HSCs being derived from the Dnmt3ab-dKO primary HSCs. This increase in abundance of Dnmt3-KO HSCs did not correlate with alterations in the quantity or proportions of committed progenitors (FIG. 6e-f). The apparent enhanced HSC activity from Dnmt3a and Dnmt3b-KO HSCs in secondary transplants was a cell autonomous mechanism as they showed higher activity on a per cell basis in colony forming capacity in vitro (FIG. 10a). PCR screening of genomic DNA from individual colonies (each derived from a single HSC) showed high efficiency of conditional deletion in clonal HSCs with >95% deletion of target alleles in serially transplanted Dnmt3-KO HSCs (FIG. 6b).

In further characterizing the cellular differences between serially-transplanted control and Dnmt3-KO HSCs, the observed HSC expansion could not be attributed to either enhanced proliferation or more resistance to apoptosis from Dnmt3-KO HSCs (FIG. 6g-h). Dnmt3-KO HSCs were actually significantly less proliferative than transplant-matched control HSCs. Serial re-purification of donor-derived HSCs 18-weeks post transplant and re-transplantation of 250 HSCs into new recipients demonstrated that the peripheral blood chimerism in Dnmt3a- and Dnmt3b-KO HSC-transplanted mice remained marginally superior to control HSCs in tertiary and quaternary transplants (FIG. 5a). However, the number of phenotypically-defined HSCs continued to increase dramatically (FIG. 5c). While Dnmt3ab-dKO HSCs failed to contribute to peripheral blood cell production past secondary transplantation, they were still able to generate substantial numbers of Dnmt3ab-dKO HSCs in the bone marrow of tertiary and quaternary recipient mice. Thus, the levels of peripheral blood cell differentiation from Dnmt3-KO HSCs did not proportionally correlate with their abundance in the bone marrow. Despite this apparent differentiation block in vivo, Dnmt3-KO HSCs were still capable of robust myleoid and lymphoid differentiation in vitro under appropriate conditions.

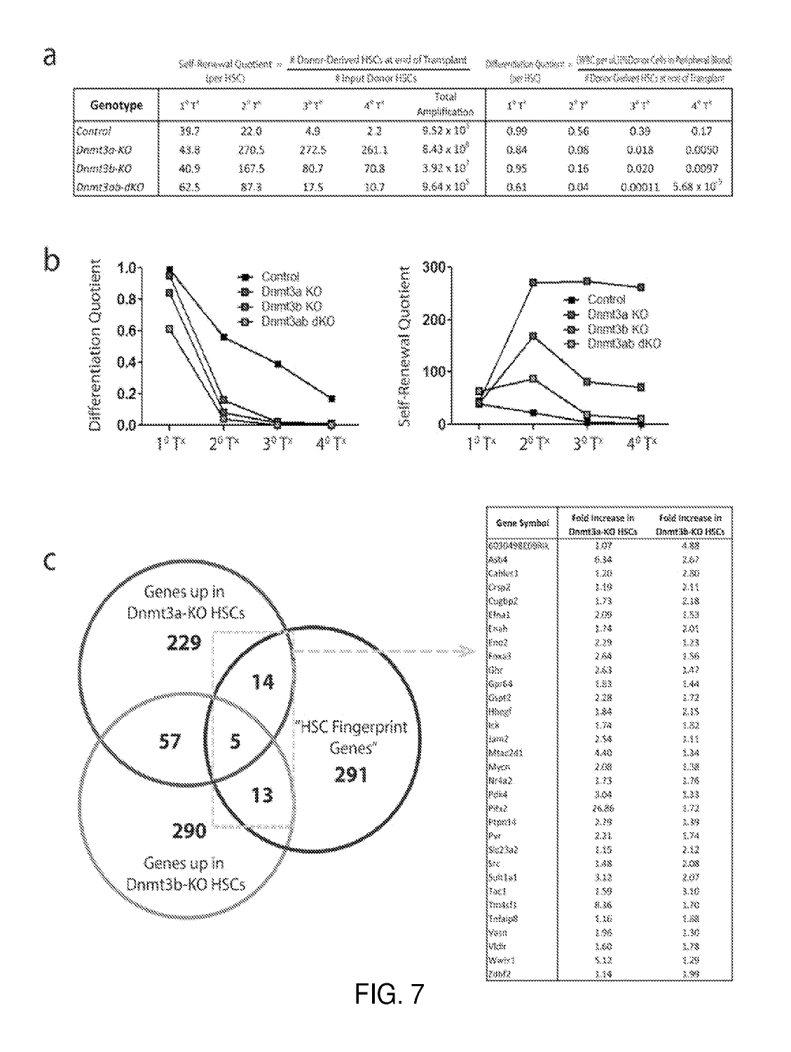

Scrutiny of multiple criteria determined that Dnmt3-KO HSCs favor self-renewal cell fate decisions (self-renewal quotient=number of donor-derived HSCs produced at the end of the transplant per original input HSC) at the expense of differentiation (differentiation quotient=16-week white blood cell count per uL blood×percentage donor CD45.2⁺ blood chimerism/number of donor-derived HSCs) on a per cell level in vivo (FIG. 7a). This differentiation deficit most rapidly appeared and was most severe in the Dnmt3ab-dKO HSCs and became progressively worse for each genotype after each round of serial transplantation (FIG. 7b). Conversely, the self-renewal quotient of Dnmt3-KO HSCs was drastically higher than control HSCs, with the Dnmt3a-KO HSCs in particular sustaining robust levels up to quaternary transplantation. This can be highlighted by calculating the total number of HSCs generated after four-rounds of transplantation from each input HSC at the beginning of the transplantation experiments. After four rounds of transplantation, control HSCs produced 9.5×10³ HSCs per each original input. In contrast, all Dnmt3-KO genotypes generated substantially more in comparison (Dnmt3a-KO=8.4×10⁸; Dnmt3b-KO=3.9×10⁷; Dnmt3ab-dKO=9.6×10⁵). These data indicate ablation of de novo DNA methylation in HSCs skews the balance between normal self-renewal and differentiation on a per-HSC level, with Dnmt3-KO HSCs influenced to self-renew and accumulate in the bone marrow rather than engage hematopoietic differentiation programs. Because wild-type whole bone marrow competitor was used in all these transplant experiments, it was not clear if the accumulation of Dnmt3-KO HSCs in the bone marrow was a compensatory mechanism to overcome their differentiation deficiency, or it this skewing of acute cell fate decisions resulted from an inability to respond to differentiation signals from the niche.

To attempt to uncover the molecular changes responsible for the HSC phenotype following Dnmt3-deletion, HSCs (CD45.2⁺SP^{KLS}CD150⁺) were purified from secondarily-transplanted mice and subject to global transcriptional profiling by microarray. Using stringent bioinformatic criteria, the inventors identified 516 and 734 unique genes differentially expressed between Dnmt3a- and Dnmt3b-KO HSCs versus control HSCs respectively. 62 unique genes were significantly upregulated in both Dnmt3-KO HSCs compared to control and 46 unique genes were similarly significantly downregulated in both Dnmt3-KO HSCs. Many of the genes upregulated in Dnmt3-KO HSCs were genes that were already highly expressed in normal HSCs, including 32 (out of 323 total) genes previously identified as “HSC fingerprint” genes (expressed specifically in HSCs but not differentiated cells; FIG. 4c) in a prior expression profiling study conducted in the lab of the inventors (Chambers et al., 2007) and other genes known to be crucial for HSC function, such as Pbx1 (Ficara et al., 2008), Stat1 (Essers et al., 2009; Baldridge et al., 2010), Nr4a2 (Sirin et al, 2010), Foxo1 (Tothova et al., 2007), Cdkn1a (Cheng et al., 2000) and Slamf1 (Kiel et al., 2005), for example.

In parallel to this, the inventors performed Digital Restriction Enzyme Analysis of Methylation (DREAM) sequencing in secondarily-transplanted B-cells to determine what methylation changes resulting from loss of Dnmt3 in the HSCs would manifest in their differentiated progeny. Analyzing the sites with significant coverage that were most hypomethylated in the Dnmt3-KO B-cells, the inventors noted that many of these genes corresponded to the “HSC fingerprint genes” that had upregulated expression in the Dnmt3-KO HSCs by microarray. Real-time PCR revealed that these genes, which were not expressed in normal B-cells, showed spurious upregulated expression in the Dnmt3-KO B-cells (and granulocytes). Cumulatively, these data indicate that Dnmt3-KO HSCs favor self-renewal over differentiation due to higher expression of “stem cell” genes and that DNA methyltransferase enzymes are required to methylate and silence the expression of these genes to permit differentiation following appropriate stimulation, as loss of these enzymes in HSCs leads to hypomethylation and aberrant expression of “HSC genes” in the differentiated progeny.

To investigate if the effects of loss of Dnmt3a in HSCs were reversible, secondarily-transplanted bone marrow was transduced with MSCV-Dnmt3a retrovirus to restore this enzyme and re-transplanted into lethally irradiated recipient mice. Analysis of long-term transplants showed that exogenous Dnmt3a expression could partially rescue HSC expansion, HSC lineage differentiation and gene expression patterns (FIG. 7). The rescue was not completely penetrant, likely due to the relative inefficiency of retroviral HSC transduction. This shows that some of the effects of loss of de novo DNA methylation in HSCs are reversible and in certain embodiments the other phenotypical aspects are able to be rescued if more efficient means of introducing exogenous Dnmt3a were used and/or if allowed to continue for longer, for example.

Finally, the inventors employed Reduced Representation Bisulfite Sequencing (RRBS) to identify the methylation changes that result from loss of Dnmt3 in serially-transplanted HSCs. The inventors performed a comparison of age-matched control HSCs versus tertiary-transplanted Dnmt3a-KO HSCs, because this genotype gave the most sustained and robust HSC amplification phenotype. Triplicate biological samples were prepared and sequenced and in some cases quality control, data analysis, and bioinformatics are performed.

Comparison of CpGs with significant coverage showed that globally there was no difference in genome-wide methylation levels of control versus Dnmt3a-KO HSCs, despite the long-term (˜12-month) absence of the DNA methyltransferase enzyme. This observation was subsequently confirmed by HPLC.

Closer scrutiny of genomic localization of differentially methylated CpGs showed that there was a significant over-representation of CpG hypermethylation in Dnmt3a-KO HSC CpG islands. Analysis of the differentially methylated regions (DMRs), defined as three consectuive CpGs (within 300 bp) with >33% change in the methylation status in the same direction, identified 869 hypermethylated DMRs in Dnmt3a-KO HSCs compared to control HSCs and 674 hypomethylated DMRs. The hypomethylated DMRs were significantly enriched in genes involved in various human hematopoietic malignancies such as Prdm16, Stat1, Ccnd1, myc, Mn1, Msi2, Ment, Erg and Runx1. Although these hypomethylation changes in these genes in Dnmt3a-KO HSCs did not necessarily correlate with gene expression differences, the observation of hypermethylated CpG islands and hypomethylated tumor suppressor genes is a characteristic hallmark of many human cancers. While Dnmt3-KO HSCs never gave rise to cancer in serial competitive transplantation, many aspects of the observed phenotype resemble a human pre-leukemic state (accumulation of undifferentiated cells, changes in proliferation and differentiation), indicating that Dnmt3 mutation may be one of the first steps in progression to leukemogenesis and that Dnmt3-KO HSCs may be sensitized to secondary transforming events.

### Significance of Certain Embodiments of the Invention

In addressing the results of Dnmt3 deletion in HSCs previously reported, the inventors obtained similar results to Tadokoro et al. using a MSCV-cre-GFP retrovirus to modulate floxed allele deletion. The drastic phenotype that was observed here was only evident upon serial transplantation of Dnmt3-KO HSCs and in certain embodiments was missed by the previous group as the bone marrow was not examined and the retroviral deletion method is not as efficient as using a genetic cre-driver system. DNA methylation is a very stable epigenetic modification and it took several rounds of serial transplantation to clearly elucidate the phenotype of HSCs following conditional ablation of the Dnmt3s. This is likely due to the time required for cell divisions to erase the methylation signatures mediated by Dnmt3s in HSCs. Particularly striking was the observation that methylation and expression changes resulting from loss of Dnmt3a and Dnmt3b in HSCs much more obviously manifest in their differentiated progeny rather than the stem cells themselves. As the hematopoietic differentiation cascade proceeds, the absence of these enzymes has marked consequences on the lineage output of HSCs and the molecular make-up of the blood cells that are produced.

In serially competitive transplantation, Dnmt3-KO HSCs were not overtly transformed and the mice were healthy. We have observed no evidence of accumulation of undifferentiated or blast-like cells in the blood or bone marrow of serially Dnmt3-KO HSC-transplanted mice and no mice have succumb to unnatural death through four-rounds of serial transfer. While Dnmt3-KO HSCs do not appear leukemic, they may resemble human pre-leukemic or myeloproliferative states. Ablation of a particular DNA methyltransferase did not affect expression of the other, (i.e. deletion of Dnmt3a in HSCs did not induce differential expression of Dnmt3b and vice versa) or Dnmt1, although this does not necessarily exclude the possibility of aberrant function of one in the absence of the other. The recent reports that Dnmt3a is mutated in >20% of human acute myeloid leukemia (AML) and 10-15% of myelodysplastic syndrome (MDS) patients, and is associated with adverse long-term clinical outcomes, indicate that de novo DNA methylation is crucial for normal HSC function and protection from oncogenic transformation.

### Example 3

### Exemplary Methods and Materials

### Mice and Transplantation

All animal procedures were IUCAC approved and conducted in accordance institutional guidelines. Mice were housed in a specific-pathogen-free facility and fed autoclaved acidified water and mouse chow ad libitum. All mice were C57Bl/6 background distinguished by CD45.1 or CD45.1 alleles. Dnmt3a^{fl/fl} and Dnmt3b^{fl/fl} mice, made by En Li, were obtained from the Beaudet lab at Baylor College of Medicine and crossed to Mx1-cre mice. For bone marrow transplantation, recipient C57Bl/6 CD45.1 were transplanted by retro-orbital injection following a split dose of 10.5 Gy of lethal irradiation. Donor HSCs (CD45.2) were competed against 2×10⁵ unfractionated WBM cells with the opposite CD45 allele (matched to the recipient). In competitive transplantation experiments, deletion of floxed alleles in Mx1-cre⁺ donor HSCs was mediated 5-6 weeks post-transplantation in primary recipients to avoid any complications loss of Dnmt3 may have on HSC homing. Conditional deletion was mediated by six intraperitoneal injections (300 μg/mouse) of polyinosinic-polycytidylic acid (pIpC; Sigma) in PBS every other day to induce an interferon response in the animals and subsequent expression of Mx1-cre in target cells. For serial HSC transplantation, WBM from transplanted recipients was isolated 18-weeks post-transplant and donor HSCs were re-purified using CD45.2⁺SP^{KLS} gating. 250 of these re-purified donor HSCs were competed against 2×10⁵ fresh CD45.1 WBM cells in new lethally irradiated recipients.

### Hematopoietic Stem Cell Purification and Flow Cytometry

Whole bone marrow (WBM) was isolated from femurs, tibias and iliac crests. SP staining was performed with Hoechst 33342 (Sigma-Aldrich, St Louis, Mo.) as previously described. Briefly, whole bone marrow was resuspended in staining media at 10⁶ cells/mL and incubated with 5 μg/mL Hoechst 33342 for 90 minutes at 37° C. For antibody staining, cells were suspended at a concentration of 10⁸ cells/mL and incubated on ice for 20 minutes with the desired antibodies.

### Plasmids and Retroviral Over-Expression

Full-length mouse Dnmt3a (Accession BC007466) and Dnmt3b (Accession BC105677) cDNAs were purchased from Open Biosystems (Huntsville, Ala., USA). Open reading frames were subcloned into MSCV-RFB-IRES-GFP vector using Gateway recombination. Briefly, Dnmt3a and Dnmt3b open reading frames were PCR amplified and inserted into the pENTR-D/TOPO vector (Invitrogen) by TOPO cloning. Correct clones were sequence verified and recombined into an MSCV-RFB-IRES-GFP vector (containing attR recombination sites) using LR clonase enzyme mixture (Invitrogen) to produce retroviral vectors. MSCV-IRES-GFP containing no open reading frame was used as a control vector in all experiments. Viruses were packaged by cotransfection with pCL-Eco (Naviaux, 1996) into 293T cells. Viral supernatants were collected 48-hours post-transfection and viral titers determined using 3T3 cells.

For retroviral transduction of hematopoietic progenitors, donor CD45.2 mice were treated with 5-fluorouracil (150 mg/kg, American Pharmaceutical Partners) six days prior to bone marrow harvest. Whole bone marrow was enriched for Sca-1+ cells using magnetic enrichment (AutoMACS, Miltenyi) and adjusted to a concentration of 5×10⁵ cells/ml in transduction medium, containing Stempro 34 (GIBCO), nutrient supplement, penicillin/streptomycin, L-glutamine (2 mM), mSCF (10 ng/ml, R&D Systems), mTPO (100 ng/ml, R&D Systems), and polybrene (4 ug/ml, Sigma). The suspension was spin-infected at 250×g at room temperature for two hours (Kotani, 1994), and cells were incubated for a further three hours at 37° C. For in vitro assays, transduced cells were cultured in fresh transduction medium for a further two days. For clonal colony-forming analysis, single HSCs (Sca-1+GFP+) were sorted into 96-well plates containing Methocult 3434 medium (Stem Cell Technologies, Vancouver, BC, Canada) supplemented with penicillin/streptomycin and cultured in vitro at 37° C. for two-weeks.

### Peripheral Blood Analysis

Peripheral blood counts were performed with a Hemavet 950. For peripheral blood analysis, mice were bled retro-orbitally, the red blood cells were lysed, and each sample was incubated with the following antibodies on ice for 20 min: CD45.1-FITC, CD45.2-APC, CD4-Pacific Blue, CD8-Pacific Blue, B220-Pacific Blue, B220-PeCy7, Mac1-PeCy7, and Gr-1-PeCy7 as previously described (Challen et al., 2009). Cells were then spun down and resuspended in a propidium iodide solution, and analysis was accomplished on live cells with an LSRII (Becton Dickinson).

### Methocult Assays and Genotyping

Single HSCs were sorted into 96-well plates containing Methocult 3434 medium (Stem Cell Technologies) supplemented with penicillin/streptomycin and cultured in vitro at 37° C. for 2 weeks. Genomic DNA was prepared from Methocult colonies with the Genomic DNA extraction kit. PCR screening of floxed allele deletion was performed with the following primers;

### Immunoflourescence

Flow cytometry-purified cells were cytospun onto microscope slides and fixed with 4% paraformaldehyde for 10 min. Cells were permeabilized with 1% Triton X-100 for 20 min and blocked with 10% goat serum for 1 h. Slides were stained with rabbit anti-Dnmt3a (sc-20703; Santa Cruz Biotechnology, Santa Cruz, Calif., USA) and goat anti-mouse Dnmt3b (sc-10235; Santa Cruz Biotechnology, Santa Cruz, Calif., USA) overnight at 4° C. (both 1:250 dilution) and counterstained with anti-rabbit-Alexa594 and anti-goat-Alexa488 secondary antibodies (both at 1:500 dilution; Invitrogen, Carlsbad, Calif., USA) for 45 min at room temperature. Slides were mounted in Vectashield+DAPI (Vector Laboratories) and images captured on a Zeiss Axioplan 2 microscope equipped with Photometrics Coolsnap HQ camera or a DeltaVision restoration microscope and Softworx imaging software.

### Cell Cycle Analysis—BrdU, Ki67 and Propidium Iodide Staining

BrdU analysis of HSCs was performed as described, with 1 mg BrdU per 6 g of mouse weight injected intraperitoneally 12 h or 3 days before analysis (Naviaux et al., 1996). For the 3-day experiment, BrdU (Sigma) was also added at 1 mg ml-1 to the drinking water. For in vitro analysis, BrdU was added at a concentration of 10 μM to the media for 12 h. HSCs were sorted into a tube containing 300,000-500,000 carrier B220+ cells before fixation, DNase treatment, and cell staining using the BrdU FITC Flow Kit (BD PharMingen). Flow cytometric analysis of cells was conducted using a FacScan flow cytometer. Analysis of all flow cytometry data was conducted using FlowJo (Treestar). Data reflect at least two independent experiments, each conducted in triplicate.

Mice received one intraperitoneal injection of BrdU (Sigma-Aldrich; 1 mg/6 g of mouse weight) and sacrificed 12 hours later. WBM was stained with antibodies to identify stem and progenitor cell compartments and then prepared for analysis of BrdU incorporation using the FITC-BrdU Flow Kit (BD Pharmingen).

For cell cycle analysis, HSCs were sorted into deionized water containing 0.1% sodium citrate and 50 lg/ml propidium iodide (PI) and analyzed with a FACScan flow cytometer (BD Biosciences).

### AnnexinV Staining

Annexin V and PI staining was used to assess cell death and apoptosis. Briefly, cells were washed twice with cold PBS and incubated at room temperature in 1× binding buffer (10 mM HEPES, 140 mM NaCl, 2.5 mM CaCl₂) containing AnnexinV-APC (BD-Pharmingen) and PI. Cells were analyzed by flow cytometry within one hour of staining.

### Quantitative Real-Time PCR

RNA was isolated from FACS-sorted cells using the RNAqueous kit (Ambion). RNA was reverse transcribed with random hexamer primers using Super Script II (Invitrogen). cDNA input was standardized and RT-PCRs were performed with Taqman master Mix (Applied Biosystems), 18 s-rRNA probe (VIC-MGB; Applied Biosystems), and a gene-specific probe (FAM-MGB; Applied Biosystems) for 60 cycles with an AbiPrism 7900HT (Applied Biosystems). Samples were normalized to 18S and fold-change determined by the ΔΔCt method.

### Microarrays

HSCs (CD45.2⁺SP^{KLS}CD150⁺) were sorted from three biological replicate pools of secondarily-transplanted mice for each genotype.

Re-sorting of intial samples confirmed purity of each sort was >98%. RNA was isolated with the RNAqueous (Ambion), treated with DNase I, and precipitated with phenol:chloroform:isoamyl alcohol. The RNA was linearly Amplified in two rounds of T7 in vitro transcription (MessageAmp, Ambion) and labeled with biotin-UTP and -CTP (Enzo Biotech) during the second amplification. Labeled RNA (20 mg) was diluted in fragmentation buffer, incubated at 94° C. for 25 min, and hybridized to Affymetrix MOE430.2 chips according to standard protocols. The raw image and intensity files were generated with GCOS 1.0 software (Affymetrix). Microarray chips passed quality control tests, including a scale factor <5, a 50 to 30 probe ratio <20, a replicate correlation coefficient >0.96, unbiased global clustering analysis, and limited RNA degradation analyzed with the 50 to 30 signal intensity ratios from chip probes. Normalization and model-based expression measurements were performed with GC-RMA. Determination of differentially expressed genes between samples was defined as fold-change >2, adjusted p value <0.05, B-statistic >0. Microarray data were further analyzed with Ingenuity Pathways Analysis (Ingenuity Systems).

### HPLC

DNA hydrolysis was performed as previously described by Song et al (Song L, James S R, Kazim L, Karpf A R. (2005) Anal Chem. 77(2):504-10. Specific method for the determination of genomic DNA methylation by liquid chromatography-electrospray ionization tandem mass spectrometry).

Briefly, one microgram of genomic DNA was first denatured by heating at 100° C. Five units of Nuclease P1 (Sigma, Cat # N8630), was added and the mixture incubated at 37° C. for 2 hours. A 1/10 volume of 1M Ammonium bicarbonate and 0.002 units of venom phosphodiesterase 1 (SIGMA, # P3243) were added to the mixture and the incubation continued for 2 hours at 37° C. Next, 0.5 units of Alkaline phosphatase (Roche, Cat #108 138) was added and the mixture incubated for 1 hour at 37 C. Before injection into an Agilent Zorbax Eclipse XDB-C₁₈ 4.6×50 mm column (3.5 μm particle size), the reactions were diluted with water to dilute out the salts and the enzymes. LC separation was performed at a flow rate of 220 μL/min. Quantification was done using a LC-ESI-MS/MS system in the multiple reaction monitoring (MRM) mode as described (Song et al).

### RRBS

RRBS libraries were prepared as described previously. (Methods 2009 48(3):226-32). Total 50-100 ng of mouse genomic DNA digested with 10 Unit MspI (NEB) which cuts at CCGG site methylation insensitively. Digested fragments were end-repaired, A-tailed and ligated to illumine adapters. End-repair and 3′ adenylation was performed in 50 μl reaction containing 4 mM 5′ methylated dCTP, 4 mM dGTP, 40 mM dATP, and 10 U of 3′ to 5′ exo-Klenow DNA polymerase (NEB) and incubated 30° C. for 20 min followed by 20 min at 37° C. Adaptor ligation was performed in 50 μl reaction containing 300 mM pre-methylated adapters and 1000 Unit T4 DNA polymerase and incubated at 16° C. overnight. Adaptor-ligated DNA was subjected to a size selection on a 3% NuSieve 3:1 agarose gel. DNA marker lanes were excised from the gel and stained with SYBR Gold (Invitrogen). 160-350 bp slices were excised from the unstained gel and purified using MinElute spin column (Qiagen). Size-selected fragments were bisulphite-treated using the EpiTect Bisulphite Kit (Qiagen) with minor modifications by adding 5 more cycles (5 min 95° C. followed by 90 min at 60° C.). After bisulphate conversion, DNA was eluted in 40 μl EB buffer and 0.8 μl DNA was used for analytical PCR reactions to determine the minimum number of PCR cycles required to get enough material for sequencing. Final PCR products were purified on MinElute columns (Qiagen) and assessed on 4-20% polyacrylamide Criterion TBE Gel (Bio-Rad) and quantified using Qubit fluorometer (Invitrogen). The libraries were sequenced on the Illumina Genome Analyzer II following the manufacturer's instructions.

### Statistics

Student's t test and 1-way ANOVAs were used for statistical comparisons where appropriate. Significance is indicated on the figures with the following convention: *p<0.05, **p<0.01, ***p<0.001. Error bars on all graphs represent the SEM.

### REFERENCES

The following references, to the extent that they provide exemplary procedural or other details supplementary to those set forth herein, are specifically incorporated herein by reference in their entirety.

### Publications

Attwood, J. T., Yung, R. L., & Richardson, B. C., DNA methylation and the regulation of gene transcription. Cell Mol Life Sci 59 (2), 241-257 (2002).
Baldridge, M. T., King, K. Y., Boles, N. C., Weksberg, D. C., & Goodell, M. A., Quiescent haematopoietic stem cells are activated by IFN-gamma in response to chronic infection. Nature 465 (7299), 793-797.
Challen, G. A., Boles, N. C., Chambers, S. M., & Goodell, M. A., Distinct hematopoietic stem cell subtypes are differentially regulated by TGF-beta1. Cell Stem Cell 6 (3), 265-278.
Chambers, S. M. et al., Hematopoietic fingerprints: an expression database of stem cells and their progeny. Cell Stem Cell 1 (5), 578-591 (2007).
Chen, T., Ueda, Y., Dodge, J. E., Wang, Z., & Li, E., Establishment and maintenance of genomic methylation patterns in mouse embryonic stem cells by Dnmt3a and Dnmt3b. Mol Cell Biol 23 (16), 5594-5605 (2003).
Cheng, T. et al., Hematopoietic stem cell quiescence maintained by p21cip1/waf1. Science 287 (5459), 1804-1808 (2000).
Dodge, J. E. et al., Inactivation of Dnmt3b in mouse embryonic fibroblasts results in DNA hypomethylation, chromosomal instability, and spontaneous immortalization. J Biol Chem 280 (18), 17986-17991 (2005).
Essers, M. A. et al., IFNalpha activates dormant haematopoietic stem cells in vivo. Nature (2009).
Ficara, F., Murphy, M. J., Lin, M., & Cleary, M. L., Pbx1 regulates self-renewal of long-term hematopoietic stem cells by maintaining their quiescence. Cell Stem Cell 2 (5), 484-496 (2008).
Kiel, M. J. et al., SLAM family receptors distinguish hematopoietic stem and progenitor cells and reveal endothelial niches for stem cells. Cell 121 (7), 1109-1121 (2005).
Kotani, H. et al., Improved methods of retroviral vector transduction and production for gene therapy. Hum Gene Ther 5 (1), 19-28 (1994).
Lei, H. et al., De novo DNA cytosine methyltransferase activities in mouse embryonic stem cells. Development 122 (10), 3195-3205 (1996).
Naviaux, R. K., Costanzi, E., Haas, M., & Verma, I. M., The pCL vector system: rapid production of helper-free, high-titer, recombinant retroviruses. J Virol 70 (8), 5701-5705 (1996).
Okano, M., Xie, S., & Li, E., Cloning and characterization of a family of novel mammalian DNA (cytosine-5) methyltransferases. Nat Genet 19 (3), 219-220 (1998).
Okano, M., Bell, D. W., Haber, D. A., & Li, E., DNA methyltransferases Dnmt3a and Dnmt3b are essential for de novo methylation and mammalian development. Cell 99 (3), 247-257 (1999).
Osawa, M., Hanada, K., Hamada, H., & Nakauchi, H., Long-term lymphohematopoietic reconstitution by a single CD34-low/negative hematopoietic stem cell. Science 273 (5272), 242-245 (1996).
Sirin, O., Lukov, G. L., Mao, R., Conneely, O. M., & Goodell, M. A., The orphan nuclear receptor Nurr1 restricts the proliferation of haematopoietic stem cells. Nat Cell Biol.
Spangrude, G. J. & Johnson, G. R., Resting and activated subsets of mouse multipotent hematopoietic stem cells. Proc Natl Acad Sci USA 87 (19), 7433-7437 (1990).
Tadokoro, Y., Ema, H., Okano, M., Li, E., & Nakauchi, H., De novo DNA methyltransferase is essential for self-renewal, but not for differentiation, in hematopoietic stem cells. J Exp Med 204 (4), 715-722 (2007).
Tothova, Z. et al., FoxOs are critical mediators of hematopoietic stem cell resistance to physiologic oxidative stress. Cell 128 (2), 325-339 (2007).
Venezia, T. A. et al., Molecular signatures of proliferation and quiescence in hematopoietic stem cells. PLoS Biol 2 (10), e301 (2004).
Wilson, A. et al., Dormant and self-renewing hematopoietic stem cells and their niches. Ann N Y Acad Sci 1106, 64-75 (2007).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of increasing expansion of stem cells, comprising the step of delivering to the cells an agent that inhibits expression of one or more DNA methyltransferases (DNMT) in the cells.

2. The method of claim 1, wherein the stem cells are hematopoietic stem cells.

3. The method of claim 1, further defined as providing one or more inhibitors of DNMTs to the cells.

4. The method of claim 1, wherein the DNMT is DNMT3a, DNMT3b, or both.

5. The method of claim 1, wherein the agent is a nucleic acid, protein, or small molecule.

6. The method of claim 5, wherein the nucleic acid is siRNA, shRNA, or miRNA.

7. The method of claim 5, wherein the protein is antibody.

8. The method of claim 4, wherein the agent is selected from the group consisting of RG 108, zebularine, decitabine, 5-azacytidine, BIX01294, 5-azadeoxycytidine, (−)-epigallocatechin-3-gallate (EGCG), 4-aminobenzoic acid derivative, a psammaplin, and an oligonucleotide.
